(19) United States
(12) Patent Application Publication
(10) Pub. No.: US 2004/0013652 A1
(43) Pub. Date: Jan. 22, 2004

(54) Treatments with autologous fibroblast

(76) Inventors: Olga Marko, Houston, TX (US); William K. Boss JR., Essex Fells, NJ (US)

Correspondence Address: FISH & RICHARDSON P.C., 45 ROCKEFELLER PLAZA, SUITE 2800, NEW YORK, NY, 10111, US

(21) Appl. No.: 10/420,437

(22) Filed: Apr. 22, 2003

Related U.S. Application Data
(60) Provisional application No. 60/379,344, filed on May 10, 2002.

(51) Int. Cl. A61K 45/00; A61K 38/39; A61K 31/715

(52) U.S. Cl. 424/93.7; 514/8; 514/54

(57) ABSTRACT

The invention provides compositions containing autologous, passaged fibroblasts and, optionally, autologous, passaged muscle cells, biodegradable acellular matrix components, and/or biodegradable acellular fillers. The invention also provides methods for making the compositions, as well as devices and methods for administering the compositions to treat conditions such as urinary incontinence, vesicoureteral reflux, and gastroesophageal reflux.

This application claims priority of U.S. Provisional Application No. 60/379,344, filed May 10, 2002. The disclosure of U.S. Provisional Application No. 60/379,344 is incorporated herein by reference in its entirety.

### TECHNICAL FIELD

This invention relates to treatment of urinary incontinence, vesicoureteral reflux, and gastroesophageal reflux.

### BACKGROUND

Urinary incontinence is an extremely prevalent condition throughout the United States. The U.S. Department of Health and Human Services reported in 1996 that 13 million people in this country suffer from urinary incontinence. The condition is far more prevalent in women than men. In the general population aged 15 to 64 years old, 10-30% of women versus 1.5-5% of men are affected. At least 50% of nursing home residents are affected and of that number, 70% are women.

Urinary incontinence can result from anatomic, physiologic, or pathologic factors. Acute and temporary incontinence are commonly caused by childbirth, limited mobility, medication side effects, and urinary tract infections. Chronic incontinence is commonly caused by birth defects, bladder muscle weakness, a blocked urethra (due to, for example, benign prostate hyperplasia or a tumor), brain or spinal cord injuries, nerve disorders (e.g., congenital and acquired disorders of muscle innervation such as amyotrophic lateral sclerosis, spina bifida, or multiple sclerosis), and pelvic floor muscle weakness.

There are several types of urinary incontinence. Stress incontinence refers to urine loss during physical activity that increases abdominal pressure (e.g., coughing, sneezing, or laughing). Urge incontinence results in urine loss with an urgent need to void and involuntary bladder contraction. Mixed incontinence includes both stress and urge incontinence. Overflow incontinence involves a constant dribbling of urine, although the bladder never completely empties. Of these types of urinary incontinence, stress, urge, and mixed incontinence account for more than 90% of cases. Overflow incontinence is more common in people with disorders that affect the nerve supply originating in the upper portion of the spinal cord and in older men with benign prostate hyperplasia.

Problems with urinary incontinence typically originate in the urethra, and often are due to incompetence of the urethral sphincteric mechanism at the neck of the bladder. With such incompetence, the resistance to urinary outflow is lowered to the point that involuntary urine loss occurs.

Vesicoureteral reflux is a related condition that also involves insufficient resistance to urinary outflow. This disorder involves inappropriate backflow of urine from the bladder into the ureter, which can be accompanied by intrarenal reflux and thus can be complicated by reflux nephropathy.

Gastroesophageal reflux disease (GERD) is a condition involving a similar mechanism, in which stomach contents back up into the esophagus. Gastric contents normally are retained in the stomach through the action of the lower esophageal sphincter, which remains tonically contracted except during swallowing. GERD occurs when this sphincter is functionally incompetent, intermittently relaxed or disrupted. Methods effective to control inappropriate urine outflow from the bladder and improper food back-up into the esophagus would be useful for treating patients with urinary incontinence, vesicoureteral reflux, and GERD.

U.S. Pat. Nos. 5,858,390, 5,665,372, 5,660,850, and 5,591,444, and co-pending U.S. application Ser. No. 09/678,047 are incorporated herein by reference in their entirety.

### SUMMARY

The invention provides methods for treating conditions such as urinary incontinence, vesicoureteral reflux, and esophageal reflux by injecting a suspension of histologically compatible, autologous, passaged fibroblasts and muscle cells. The invention provides methods for rendering the fibroblasts and muscle cells substantially free of immunogenic proteins present in the culture medium. The invention also provides compositions containing autologous fibroblasts and muscle cells. Compositions of the invention also can contain bulking agents and/or biodegradable acellular matrix components. The compositions can be injected into a subject to treat conditions such as urinary incontinence, vesicoureteral reflux, or esophageal reflux. The invention further provides devices for injecting such compositions.

A suspension of histologically compatible, autologous fibroblasts and muscle cells that is administered by periurethral or transurethral injection can increase pressure on the urethra and compress the urethral lumen, thus alleviating urinary incontinence by enhancing urethral resistance to the flow of urine. Similarly, a suspension of such cells that is administered by injection into tissues adjacent to the ureteral orifice can increase support behind a refluxing intravesical ureter, thus alleviating vesicoureteral reflux by providing resistance to urinary reflux. GERD can be treated by injecting a suspension of cells into tissues adjacent to the lower esophageal sphincter. The injected fibroblasts typically are fibroblasts (e.g., dermal fibroblasts) derived from the culture of a biopsy specimen taken from the subject. Muscle cells (e.g., cells from striated muscle) also can be obtained from the subject. Extensive washing of the cells results in the removal of essentially all serum-derived proteins that would be immunogenic upon administration of the cell suspension to the subject.

The invention is based on the discoveries that autologous cells are ideal for use as a bulk-enhancing agent to treat conditions such as urinary incontinence and vesicoureteral reflux, and that an abundant supply of such cells can be obtained by culturing a biopsy specimen taken from the subject several weeks prior to treatment. The invention is further based on the discovery that untoward immune responses (e.g., inflammatory immune responses) in a subject can be avoided by removing antigenic proteins from the autologous cultured cells prior to their administration to the subject.

In one aspect, the invention features a composition for repairing tissue that has degenerated in a subject as a result of a disease, disorder, or defect in the subject. The composition can contain autologous, passaged fibroblasts and autologous, passaged muscle cells, and can be substantially free of culture medium serum-derived proteins. The disease, disorder, or defect can be associated with urinary incontinence, vesicoureteral reflux, or gastroesophageal reflux. The autologous fibroblasts can be from the gums, palate, skin, lamina propria, connective tissue, bone marrow, or adipose tissue of the subject. The autologous muscle cells can be striatal muscle cells (e.g., striatal muscle cells from the tongue, palatoglossus, temporalis muscle, soleus, gastrocnemius, or stemocleidomastoid muscle of the subject). The autologous muscle cells can also be smooth muscle cells.

The composition can further contain a biodegradable acellular matrix, wherein the fibroblasts and muscle cells are integrated within and on the matrix. The matrix, prior to combination with the fibroblasts and muscle cells, can contain one or more substances selected from the group consisting of collagen, glycosaminoglycans, gelatin, polyglycolic acid, cat gut, demineralized bone, hydroxyapatite, and anorganic bone. The collagen can be, for example, bovine collagen, porcine collagen type I, or porcine collagen type III. The fibroblasts and muscle cells integrate on and within the matrix so as to substantially fill the space on and within the matrix available for cells.

In another aspect, the invention features a method for making a composition for repairing tissue that has degenerated in a subject as a result of a disease, disorder, or defect in the subject. The method can involve: (a) providing a biopsy of fibroblast-containing tissue from the subject; (b) separating autologous fibroblasts from the biopsy; (c) culturing the autologous fibroblasts under conditions that produce autologous fibroblasts that are substantially free of culture medium serum-derived proteins; (d) exposing the cultured autologous fibroblasts to conditions that result in suspension of the fibroblasts; (e) providing a biopsy of muscle tissue from the subject; (f) culturing autologous muscle cells isolated from the muscle tissue under conditions that result in muscle cells that are substantially free of culture medium serum-derived proteins; (g) exposing the cultured autologous muscle cells to conditions that result in suspension of the muscle cells; and (h) combining the fibroblasts with the muscle cells. The disease, disorder, or defect can be associated with urinary incontinence, vesicoureteral reflux, or gastroesophageal reflux.

The step of providing a biopsy of fibroblast containing tissue can involve providing a biopsy from the gums, palate, skin, lamina propria, connective tissue, bone marrow, or adipose tissue of the subject. The step of providing a biopsy of muscle tissue can involve providing a biopsy from the tongue, palatoglossus, temporalis muscle, soleus, gastrocnemius, or stemocleidomastoid muscle of the subject. Culturing of the fibroblasts or the muscle cells can involve: (1) incubation in a culture medium containing between 0.1% and about 20% human or non-human serum, followed by (2) incubation in a serum-free culture medium. Culturing of the fibroblasts or the muscle cells can involve incubation in serum-free medium. Culturing of the fibroblasts or the muscle cells can be in a medium containing one or more reagents that prevents the growth of mycoplasma (e.g., tylosin, plasmocin, mycoplasma removal agent, gentamicin, ciprofloxacine, alatrofloxacine, azithromycin, or tetracycline). The conditions that result in suspension of the fibroblasts or the muscle cells can include a proteolytic enzyme.

In another aspect, the invention provides a method for making a composition for repairing tissue that has degenerated in a subject as a result of a disease, disorder, or defect in the subject. The method can involve: (a) providing autologous, passaged fibroblasts and autologous, passaged muscle cells; (b) providing a biodegradable acellular matrix; and (c) incubating the fibroblasts and muscle cells with the biodegradable acellular matrix such that the fibroblasts and muscle cells integrate on and within the biodegradable acellular matrix, wherein the incubation results in a composition for repairing tissue, and wherein the conditions of the incubation are such that the composition is substantially free of culture medium serum-derived proteins. The disease, disorder, or defect can be associated with urinary incontinence, vesicoureteral reflux, or gastroesophageal reflux.

The biodegradable acellular matrix, prior to combination with the suspensions of fibroblasts and muscle cells, can contain one or more substances selected from the group consisting of collagen, glycosaminoglycans, gelatin, polyglycolic acid, cat gut, demineralized bone, hydroxyapatite, and anorganic bone. The collagen can be bovine collagen, porcine collagen type I, or porcine collagen type III. The fibroblasts and the muscle cells can be combined prior to the incubation. The fibroblasts and muscle cells can be added separately to the incubation. Alternatively, the incubating can involve: (1) culturing in culture medium containing between 0.1% and about 20% human or non-human serum, followed by (2) culturing in serum-free culture medium. The incubating can involve culturing in serum-free medium. The fibroblasts and muscle cells can integrate within the biodegradable acellular matrix to substantially fill the space on and within the biodegradable acellular matrix available for cells.

The step of providing autologous, passaged fibroblasts and autologous, passaged muscle cells can involve: (a) providing a biopsy of fibroblast-containing tissue from the subject; (b) separating autologous fibroblasts from the biopsy; (c) culturing the fibroblasts; (d) suspending the fibroblasts; (e) providing a biopsy of muscle tissue from the subject; (f) isolating muscle cells from the muscle tissue; (g) culturing the muscle cells; and (h) suspending the muscle cells. The step of providing a biopsy of fibroblast-containing tissue can involve providing a biopsy from the gums, palate or skin of the subject. The step of providing a biopsy of muscle tissue can involve providing a biopsy from the tongue, palatoglossus, temporalis muscle, soleus, gastrocnemius, or sternocleidomastoid muscle of the subject. Culturing of the fibroblasts and the muscle cells can be in a medium containing a reagent that prevents the growth of mycoplasma (e.g., tylosin, mycoplasma removal agent, plasmocin, gentamicin, ciprofloxacine, alatrofloxacine, azithromycin, or tetracycline).

In yet another aspect, the invention provides a method for repairing tissue in a subject. The method can involve: (a) providing a composition of the invention; (b) identifying a site of tissue defect or tissue degeneration in the subject; and (c) placing the composition at the site so that the tissue defect or degeneration is repaired. The tissue defect or tissue degeneration can result in urinary incontinence, vesicoureteral reflux, or gastroesophageal reflux. The autologous fibroblasts can be from the gums, palate, skin, lamina propria, connective tissue, bone marrow, or adipose tissue of the subject. The autologous muscle cells can be from the tongue, palatoglossus, temporalis muscle, solcus, gastrocnemius, or stemocleidomastoid muscle of the subject.

In another aspect, the invention provides a method for repairing a tissue defect in a subject. The method can involve: (a) providing a pharmaceutical composition containing (1) autologous, passaged fibroblasts, (2) autologous, passaged muscle cells, and (3) a pharmaceutically acceptable carrier thereof; wherein the pharmaceutical composition is substantially free of culture medium serum-derived proteins; (b) identifying in the subject a site of tissue defect or tissue degeneration associated with a disorder selected from the group consisting of urinary incontinence, vesicoureteral reflux, and gastroesophageal reflux; (c) injecting an amount of the pharmaceutical composition adjacent to the site of tissue defect or degeneration, wherein the injecting results in repair of the tissue defect or degeneration.

The injecting can involve injecting a volume of the pharmaceutical composition into the urethra, or tissue adjacent to the urethra, of the subject such that the urethral lumen is compressed. The injecting can involve injecting a volume of the pharmaceutical composition into tissue adjacent to a ureteral orifice of the subject such that the orifice is compressed. The injecting can involve injecting a volume of the pharmaceutical composition into the tissue adjacent to the lower esophageal sphincter of the subject such that the esophagus is compressed.

The step of providing a pharmaceutical composition can involve: (a) providing a biopsy of fibroblast containing tissue from the subject; (b) separating fibroblasts from the biopsy so as to provide fibroblasts substantially free of extracellular matrix and non-fibroblast cells; (c) culturing the fibroblasts under conditions that produce fibroblasts that are substantially free of culture medium serum-derived proteins; (d) exposing the passaged fibroblasts to conditions that result in suspension of the fibroblasts; (e) providing a muscle tissue biopsy from the subject; (f) isolating muscle cells from the muscle tissue; (g) culturing the muscle cells under conditions that produce muscle cells that are substantially free of culture medium serum-derived proteins; (g) exposing the muscle cells to conditions that result in suspension of muscle cells; and (h) combining the fibroblast suspension with the muscle cell suspension and a pharmaceutically acceptable carrier to form the pharmaceutical composition.

The biopsy of fibroblast containing tissue can be taken from the gums, palate, or skin of the subject. The muscle tissue biopsy can be taken from the tongue, palatoglossus, temporalis muscle, soleus, gastrocnemius, or sternocleidomastoid muscle of the subject. Culturing of the fibroblasts or the muscle cells can involve: (1) culturing in a medium containing between 0.1% and about 20% human or non-human serum, followed by (2) culturing in a serum-free medium. Culturing of the fibroblasts or the muscle cells can involve culturing in serum-free medium. The conditions that result in suspension of the fibroblasts or muscle cells can include a proteolytic enzyme.

The invention also features an injectable composition for repairing tissue that has degenerated in a subject as a result of a disease, disorder, or defect in the subject. The injectable composition can contain: (a) autologous, passaged fibroblasts and autologous, passaged muscle cells, wherein the fibroblasts and the muscle cells are substantially free of culture medium serum-derived proteins; and (b) a biodegradable acellular injectable filler. The autologous fibroblasts can be from the gums, palate, skin, lamina propria, connective tissue, bone marrow, or adipose tissue of the subject. The autologous muscle cells can be from the tongue, palatoglossus, temporalis muscle, soleus, gastrocnemius, or sternocleidomastoid muscle of the subject. The biodegradable acellular injectable filler, prior to combination with the fibroblasts and muscle cells, can contain one or more substances selected from the group consisting of: (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from the subject. The concentration of autologous collagen fibers in the injectable dispersion can be at least 24 mg/ml. The collagen can be bovine collagen (e.g., reconstituted bovine collagen fibers cross-linked with glutaraldehyde). The ratio of sodium chloride solution and the aliquot of serum can be 1:1 by volume. The sodium chloride solution can contain 0.9% sodium chloride by volume.

In yet another aspect, the invention features a method for making an injectable composition for repairing tissue that has degenerated in a subject as a result of a disease, disorder, or defect in the subject. The method can involve: (a) providing autologous, passaged fibroblasts and autologous, passaged muscle cells, wherein the fibroblasts and the muscle cells are substantially free of culture medium serum-derived proteins; (b) providing a biodegradable acellular filler; and (c) combining the autologous, passaged fibroblasts, the autologous, passaged muscle cells, and the biodegradable acellular filler. The disease, disorder, or defect can be associated with urinary incontinence, vesicoureteral reflux, gastroesophageal reflux, defects of an oral mucosa, trauma to an oral mucosa, periodontal disease, diabetes, cutaneous ulcers, venous stasis, scars of skin, or wrinkles of skin.

The step of providing autologous, passaged fibroblasts and autologous, passaged muscle cells can involve: (a) providing a biopsy of fibroblast-containing tissue from the subject; (b) separating autologous fibroblasts from the biopsy; (c) culturing the autologous fibroblasts under conditions that result in fibroblasts that are substantially free of culture medium serum-derived proteins; (d) exposing the incubated autologous fibroblasts to conditions that result in suspension of the fibroblasts; (e) providing a biopsy of muscle tissue from the subject; (f) isolating muscle cells from the muscle tissue biopsy; (g) culturing the muscle cells under conditions that result in muscle cells that are substantially free of culture medium serum-derived proteins; and (h) exposing the muscle cells to conditions that result in suspension of the muscle cells.

The step of providing a biopsy of fibroblast containing tissue can involve providing a biopsy from the gums, palate, skin, lamina propria, connective tissue, bone marrow, or adipose tissue of the subject. The step of providing a biopsy of muscle tissue can involve providing a biopsy from the tongue, palatoglossus, temporalis muscle, soleus, gastrocnemius, or stemocleidomastoid muscle of the subject. Culturing of the fibroblasts or the muscle cells comprises: (1) culturing in a medium containing between 0.1% and about 20% human or non-human serum, followed by (2) culturing in a serum-free medium. Culturing of the fibroblasts or the muscle cells can involve culturing in serum-free medium. Culturing of the fibroblasts or the muscle cells can be in a medium containing a reagent that prevents the growth of mycoplasma (e.g., tylosin, plasmocin, mycoplasma removal agent, gentamicin, ciprofloxacine, alatrofloxacine, azithromycin, and tetracycline). The conditions that result in suspension of the fibroblasts or muscle cells can include a proteolytic enzyme.

The biodegradable acellular filler, prior to combination with the fibroblasts and the muscle cells, can contain one or more substances selected from the group consisting of: (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from the subject. The concentration of autologous collagen fibers in the injectable dispersion can be at least 24 mg/ml. The collagen can be bovine collagen (e.g., bovine collagen fibers cross-linked with glutaraldehyde). The ratio of sodium chloride solution and the aliquot of serum can be 1:1 by volume. The sodium chloride solution can contain 0.9% sodium chloride by volume.

In another aspect, the invention features a method for repairing tissue that has degenerated in a subject as a result of a disease, disorder, or defect in the subject. The method can involve injecting an effective amount of the composition of the invention into the subject at the site of the degeneration so that the tissue is repaired. The injecting can involve injecting a volume of the composition into the urethra or tissue adjacent to the urethra of the subject such that the urethral lumen is compressed. The injecting can involve injecting a volume of the composition into the tissue adjacent to the ureteral orifice of the subject such that the orifice is compressed. The injecting can involve injecting a volume of the composition into the tissue adjacent to the lower esophageal sphincter of the subject such that the esophagus is compressed.

The biodegradable acellular injectable filler, prior to combination with the fibroblasts and muscle cells, can contain one or more substances selected from the group consisting of: (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from the subject. The collagen can be bovine collagen.

In yet another aspect, the invention features a method for repairing tissue that has degenerated in a subject as a result of a disease, disorder, or defect in the subject. The method can involve the steps of: (a) injecting autologous, passaged fibroblasts into the subject at a site of tissue degeneration, wherein the fibroblasts are substantially free of culture medium serum-derived proteins; (b) injecting autologous, passaged muscle cells into the subject at a site of a tissue defect or desired tissue augmentation, wherein the muscle cells are substantially free of culture medium serum-derived proteins; and (c) injecting a biodegradable, acellular filler into the site, wherein the filler is substantially free of culture medium serum-derived proteins. Each of the injecting steps (a)-(c) can involve injecting into the urethra or tissue adjacent to the urethra of the subject, wherein the method results in compression of the urethral lumen. Each of the injecting steps (a)-(c) can involve injecting into the tissue adjacent to a ureteral orifice of the subject, wherein the method results in compression of the orifice. Each of the injecting steps (a)-(c) can involve injecting into the tissue adjacent to the lower esophageal sphincter of the subject, wherein the method results in compression of the esophagus. The disease, disorder, or defect can involve defects of an oral mucosa, trauma to an oral mucosa, periodontal disease, diabetes, cutaneous ulcers, venous stasis, scars of skin, or wrinkles of skin.

The autologous fibroblasts can be from the gums, palate, skin, lamina propria, connective tissue, bone marrow, or adipose tissue of the subject. The autologous muscle cells can be from the tongue, palatoglossus, temporalis muscle, soleus, gastrocnemius, or sternocleidomastoid muscle of the subject. The fibroblasts and muscle cells can be injected simultaneously. The fibroblasts, muscle cells, and biodegradable acellular filler can be injected simultaneously. The fibroblasts and muscle cells can be injected separately. The fibroblasts and muscle cells can be injected separately from the biodegradable acellular filler. The duration between injecting the fibroblasts and muscle cells into the subject and injecting the biodegradable acellular filler into the subject can be about two weeks.

The biodegradable acellular filler, prior to combination with the fibroblasts and the muscle cells, can contain one or more substances selected from the group consisting of: (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from the subject. The concentration of autologous collagen fibers in the injectable dispersion can be at least 24 mg/ml. The collagen can be bovine collagen (e.g., reconstituted bovine collagen fibers cross-linked with glutaraldehyde). The ratio of sodium chloride solution to the aliquot of serum can be 1:1 by volume. The sodium chloride solution can contain 0.9% sodium chloride by volume. The the ratio of autologous, passaged fibroblasts and autologous, passaged muscle cells to biodegradable, biodegradable acellular filler can be approximately 1:1 by volume.

In another aspect, the invention features a device for repairing tissue that has degenerated in a subject as a result of a disease, disorder, or defect in the subject. The device can contain: (a) a hypodermic syringe having a syringe chamber, a piston disposed therein, and an orifice communicating with the chamber; and (b) a suspension containing autologous, passaged fibroblasts, autologous, passaged muscle cells, and a pharmaceutically acceptable carrier, wherein the suspension is substantially free of culture medium serum-derived proteins, and wherein the suspension is disposed within the chamber.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DETAILED DESCRIPTION

The invention provides methods for treating conditions such as urinary incontinence, vesicoureteral reflux, and esophageal reflux. These methods involve administering to a subject a composition that contains histologically compatible, autologous, passaged fibroblasts. The composition can also contain muscle cells (e.g., histocompatible or autologous muscle cells), and can further contain biodegradable acellular matrix components and/or a bulking agent (e.g., a biodegradable acellular filler). The invention provides methods for rendering the injected cells substantially free of immunogenic proteins that may be present in the culture medium. The invention also provides compositions containing autologous fibroblasts with and without muscle cells, biodegradable acellular matrix components, and/or bulking agents for administration to a subject with a condition such as urinary incontinence, vesicoureteral reflux, or esophageal reflux. The invention further provides methods for making and devices for injecting such compositions.

### 1. Compositions Containing Autologous Fibroblasts

The invention provides compositions that are useful for treating conditions such as urinary incontinence, vesicoureteral reflux, and esophageal reflux. Compositions of the invention contain autologous, passaged fibroblasts that are substantially free of immunogenic proteins (e.g., culture medium serum-derived proteins). As used herein, the term “autologous” refers to cells removed from a donor and administered to a recipient, wherein the donor and recipient are the same individual. As used herein, cells (e.g., autologous passaged fibroblasts) that are “substantially free of culture medium serum-derived proteins” are cells in which the fluid surrounding the cells into which the cells are incorporated contains less than 0.1% (e.g., less than 0.05%, less than 0.01%, less than 0.005%, or less than 0.001%) of xenogeneic or allogeneic serum contained in the tissue culture medium in which the cells were cultured. Similarly, a composition that is ““substantially free of culture medium serum-derived proteins” is a composition in which fluid surrounding the cells in the composition contains less than 0.1% (e.g., less than 0.05%, less than 0.01%, less than 0.005%, or less than 0.001%) of xenogeneic or allogeneic serum contained in the tissue culture medium in which the cells were cultured.

Fibroblasts can be from any mammalian species (e.g., humans, non-human primates, dogs, cows, horses, pigs, sheep, cats, rabbits, mice, rats, guinea pigs, hamsters, or gerbils) provided that the cells are autologous. Autologous human fibroblasts are particularly useful. It is noted that when animals are inbred and are thus isogenic (e.g., as in same laboratory strains of animals such as rats and mice), “autologous” can mean from derived another individual of the same species.

Compositions can contain any undifferentiated mesenchymal cell that can be expanded in culture. Fibroblasts isolated from dermal tissue are particularly useful because they can be readily obtained and expanded, and because they are a cell type normally present in tissues adjacent to the urethral sphincter, the ureteral orifice, and the lower esophageal sphincter. Autologous dermal fibroblasts can be obtained from, for example, a biopsy of the gums, palate, or skin of the subject. The dermis is located just beneath the epidermis, and typically has a thickness that ranges from 0.5 to 3 mm. The predominant cellular constituents of the dermis are fibroblasts and macrophages, although adipose cells and muscle fibers also can be present. In addition to the dermis, fibroblasts can be obtained from, without limitation, fascia, lamina propria, the bulbar area of hair follicles, bone marrow, or any source of connective tissue. In addition, fibroblasts can be derived from undifferentiated mesenchymal cells. Any suitable method for culturing and differentiating undifferentiated mesenchymal cells can be used, including those methods known in the art.

Due to the phenomenon of allograft rejection, which is well known to transplant surgeons and immunologists, it is essential that the cultured fibroblasts be histocompatible with the recipient. Histocompatibility can be ensured by obtaining a biopsy from the subject to be treated. It is understood, however, that such cells also can be obtained from an identical twin of the subject, or from an individual that is identical at the major histocompatibility complex (MHC) with the subject. Fibroblasts from a biopsy specimen can be cultured such that the resulting cells are substantially free of culture medium serum-derived proteins, which also reduces the ability of the cells to activate an untoward immune response in the subject. To generate a composition of the present invention, a fibroblast culture can be initiated from, for example, a full thickness (e.g., 1-5 mm, or more than 5 mm if enough tissue is available) dermal biopsy specimen of the gums, palate, or skin of a subject suffering from tissue degeneration (e.g., urinary incontinence, vesicoureteral reflux, or GERD). Such a biopsy specimen can be obtained using, for example, a punch biopsy procedure. Biopsies of the dermis or lamina propria also are particularly useful for obtaining autologous fibroblasts. Skin biopsies can be taken from skin, for example, behind the ear.

Before initiation of the culture, the biopsy can be washed repeatedly with antibiotic and antifungal agents. A suitable “wash medium” can contain, for example, tissue culture medium such as Dulbecco's Modified Eagle's Medium (DMEM) and some or all of the followingagents: gentamicin, amphotericin B (Fungizone®), Mycoplasma removal agent (MRA; Dianippon Pharmaceutical Company, Japan), plasmocin, and tylosin (available from, for example, Serva, Heidelberg, Germany). Gentamicin can be used at a concentration of 10 to 100 μg/ml (e.g., 25 to75 μg/ml, or about 50 μg//ml). Amphotericin B can be used at a concentration of 0.5 to 12.5 μg/ml (e.g., 1.0 to 10.0 μg/ml, or about 2.5 μg/ml). MRA can be used at a concentration of 0.1 to 1.5 μg/ml (e.g., 0.25 to 1.0 μg/ml, or about 0.5 μg/ml). Plasmocin can be used at a concentration of 1 to 50 μg/ml (e.g., 10 to 40 μg/ml, or about 25 μg/ml). Tylosin can be used at a concentration of 0.012 to 1.2 mg/ml (e.g., 0.06 to 0.6 mg/ml, or about 0.12 mg/ml).

If desired, sterile microscopic dissection can be used to separate dermal tissue in a biopsy from keratinized tissue-containing epidermis and subcutaneous adipocyte-containing subcutaneous tissue. The biopsy specimen then can be separated into small pieces using, for example, a scalpel or scissors to finely mince the tissue. In some embodiments, the small pieces of tissue are digested with a protease (e.g., collagenase, trypsin, chymotrypsin, papain, or chymopapain). Digestion with 200-1000 U/ml of collagenase type II for 30 minutes to 24 hours is particularly useful. If enzymatic digestion is used, cells can be collected by centrifugation and plated in tissue culture flasks.

If the tissue is not subjected to enzymatic digestion, minced tissue pieces can be individually placed onto the dry surface of a tissue culture flask and allowed to attach for between about 2 and about 10 minutes. A small amount of medium can be slowly added so as not to displace the attached tissue fragments. In the case of digested cells, the cells can be suspended in culture medium and placed in one or more flasks. After about 48-72 hours of incubation, flasks can be fed with additional medium. When a T-25 flask is used to start the culture, the initial amount of medium typically is about 1.5-2.0 ml. The establishment of a cell line from the biopsy specimen can take between about 2 and 3 weeks, at which time the cells can be removed from the initial culture vessel for expansion.

During the early stages of the culture, it is desirable that the tissue fragments remain attached to the culture vessel bottom. Fragments that detach can be reimplanted into new vessels. The fibroblasts can be stimulated to grow by a brief exposure to EDTA-trypsin, according to standard techniques. Such exposure to trypsin is too brief to release the fibroblasts from their attachment to the culture vessel wall. Immediately after the cultures become established and are approaching confluence, samples of the fibroblasts can be processed for frozen storage in, for example, liquid nitrogen. Any suitable method for freezing cells can be used, including any of the numerous methods that are known in the art for successfully freezing cells for later use. The freezing and storage of early rather than late passage fibroblasts is preferred because the number of passages in cell culture of normal human fibroblasts is limited.

Fibroblasts can be frozen in any freezing medium suitable for preserving fibroblasts (e.g., any commercially available freezing medium). A medium consisting of about 70% (v/v) growth medium, about 20% (v/v) fetal bovine serum (FBS) and about 10% (v/v) dimethylsulfoxide (DMSO) is particularly useful. The DMSO also can be replaced with, for example, glycerol. Thawed cells can be used to initiate secondary cultures for the preparation of additional suspensions for later use in the same subject, thus avoiding the inconvenience of obtaining a second specimen.

Any tissue culture technique that is suitable for the propagation of dermal fibroblasts from biopsy specimens can be used to expand the cells. Useful techniques can be found in, for example, R. I. Freshney, Ed., Animal Cell Culture: A Practical Approach, (IRL Press, Oxford, England, 1986) and R. I. Freshney, Ed., Culture of Animal Cells: A Manual of Basic Techniques, (Alan R. Liss & Co., New York, 1987).

Cell culture medium can be any medium suited for the growth of primary fibroblast cultures. The medium can be supplemented with human or non-human serum (e.g., autologous human serum, non-autologous human A/B serum, horse serum, or fetal bovine serum (FBS)) to promote growth of the fibroblasts. When included in the medium, serum typically is in an amount between about 0.1% and about 20% v/v (e.g., between 0.5% and 19%, between 1% and 15%, or between 5% and 12%). Higher concentrations of serum also can be used to promote faster growth of the fibroblasts. A particularly useful medium contains glucose DMEM that is supplemented with about 2 mM glutamine, about 10 mg/L sodium pyruvate, about 10% (v/v) FBS, and antibiotics (often called “complete medium”), wherein the concentration of glucose ranges from about 1,000 mg/L to about 4,500 mg/L. Fibroblasts also can be expanded in serum-free medium. In this way, the fibroblasts are never exposed to xenogeneic or allogeneic serum proteins and do not require the extra culturing in serum-free medium that is carried out when the fibroblasts are expanded in medium that contains non-autologous serum.

Growth medium used to culture fibroblasts can be supplemented with antibiotics to prevent contamination of the cultures by, for example, bacteria, fungus, yeast, and mycoplasma. Mycoplasma contamination is a frequent and particularly vexatious problem in tissue culture. In order to prevent or minimize mycoplasma contamination, an agent such as tylosin can be added to the culture growth medium. The medium can be further supplemented with one or more antibiotics/antimycotics (e.g., gentamicin, ciprofloxacine, alatrofloxacine, azithromycin, MRA, plasmocin, and tetracycline). Tylosin can be used at a concentration of 0.006 to 0.6 mg/ml (e.g., 0.01 to 0.1 mg/ml, or about 0.06 mg/ml). Gentamicin can be used at a concentration of 0.01 to 0.1 mg/ml (e.g., 0.03 to 0.08 mg/ml, or about 0.05 mg/ml). Ciprofloxacine can be used at a concentration of 0.002 to 0.05 mg/ml (e.g., 0.005 to 0.03 mg/ml, or about 0.01 mg/ml). Alatrofloxacine can be used at a concentration of 0.2 to 5.0 μg/ml (e.g., 0.5 to 3.0 μg/ml, or about 1.0 μg/ml). Azithromycin can be used at a concentration of 0.002 to 0.05 mg/ml (e.g., 0.005 to 0.03 mg/ml, or about 0.01 mg/ml). MRA can be used at a concentration of 0.1 to 1.5 μg/ml (e.g., 0.2 to 1.0 μg/ml, or about 0.75 μg/ml). Plasmocin can be used at a concentration of 1 to 50 μg/ml (e.g., 10 to 40 μg/ml, or about 25 μg/ml). Tetracycline can be used at a concentration of 0.004 to 0.1 mg/ml (e.g., 0.008 to 0.05 mg/ml, or about 0.02 mg/ml). The antibiotics can be present for the whole period of the culture or for a portion of the culture period.

Mycoplasma contamination can be assayed by an agar culture method using a system such as, for example, mycoplasma agar plates that are available from bioMérieux (Marcy l'Etiole, France) or can be prepared in house, and by PCR. The American Type Culture Collection (ATCC, Manassas, Va.) also markets a PCR “Mycoplasma Detection Kit”. Culture medium containing tylosin (0.06 mg/ml), gentamicin (0.1 mg/ml), ciprofloxacine (0.01 mg/ml), alatrofloxacine (1.0 μg/ml), azithromycin (0.01 mg/ml), and tetracycline (0.02 mg/ml) is particularly useful for preventing mycoplasma contamination. Another agent that can be useful in preventing mycoplasma contamination is a derivative of 4-oxo-quinoline-3-carboxylic acid (OQCA), which is commercially available as “Mycoplasma Removal Agent” from, for example, ICN Pharmaceuticals, Inc. (Costa Mesa, Calif.). This agent typically is used at a concentration of 0.1 to 2.5 mg/ml (e.g., 0.2 to 2.0 mg/ml, or 0.5 mg/ml). The antibiotic mixture or other agents can be present in the fibroblast cultures for the first two weeks after initiation. After two weeks of culture, antibiotic containing medium typically is replaced with antibiotic-free medium. Once a sufficient number of cells are present in the culture (e.g., when the cells are 70%-90% confluent), they can be tested for mycoplasmal, bacterial and fungal contamination. Only cells with no detectable contamination are useful in methods of the invention.

Autologous fibroblasts can be passaged into new flasks by trypsinization. For expansion, individual flasks can be split at a ratio of, for example, 1:3 to 1:5. Triple bottom, T-150 flasks having a total culture area of 450 cm² are suitable for expanding fibroblasts. A triple bottom T-150 flask can be seeded with, for example, about 1×10⁶ to about 3×10⁶ cells, depending on the size of the cells. Such a flask typically has a capacity to yield about 8×10⁶ to about 1.0×10⁷ cells. When the capacity of the flask is reached, which can require about 5-7 days of culture, the growth medium can be replaced by serum-free medium. The cells typically are incubated between about 30° C. and about 37.5° C. for at least 4 hours (e.g., overnight or about 18 hours). Incubation of the cells in serum free medium can substantially remove proteins derived from the non-autologous serum (e.g., FBS) added to the culture medium, which if present in a composition injected into a subject, could elicit an undesirable immune response. Serum-free medium can contain, for example, glucose DMEM supplemented with about 2 mM glutamine, with or without about 110 mg/L sodium pyruvate, wherein the concentration of glucose can range from approximately 1,000 mg/L to about 4,500 mg/L. A glucose concentration of approximately 4,500 mg/L is particularly useful. The serum-free medium also can contain one or more of the above-described antibiotics.

At the end of the incubation in serum free medium, the cells can be removed from the tissue culture flasks using, for example, trypsin-EDTA. Prior to administration to a subject, fibroblasts typically are washed 2 to 4 times in medium that is serum-free and phenol red-free, or in saline. Cells can be washed by centrifugation and resuspension, and then suspended for injection in an equal volume of injectable isotonic solution that has an appropriate physiological osmolarity and is substantially pyrogen and foreign protein free. Isotonic saline is a particularly useful isotonic solution. Five triple bottom T-150 flasks, grown to capacity, can yield about 3.5×10⁷ to about 7×10⁷ cells, which are sufficient to make up about 1.2 ml to about 1.4 ml of suspension. A pharmaceutically acceptable carrier can then be added to the passaged autologous fibroblasts to form a pharmaceutical composition. The phrase “pharmaceutically acceptable” refers to molecular entities and compositions that are not deleterious to cells, are physiologically tolerable, and do not typically produce an allergic or similar untoward reaction, such as gastric upset, dizziness and the like, when administered to a human. Such compositions include physiologically acceptable diluents of various buffer content (e.g., Tris-HCl, acetate, phosphate), pH and ionic strength.

Prior to administration, fibroblasts can be incubated with an activating compound such as, for example, ascorbic acid, ascorbyl palmitate, linoleic acid, C-Med 100® (Optigene-X LLC, Shrewsbury, N.J.), CoEnzyme Q-10, glycolic acid, L-hydroxy acid, L-lipoic acid, calcium monophosphate, or other stimulatory additives such as growth factors. Incubation with such compounds can stimulate the fibroblasts and enhance their collagen production. One or more activating compounds also can be administered to a subject along with a composition containing autologous, passaged fibroblasts. Alternatively, administration of an activating compound in the absence of passaged fibroblasts can be used to stimulate fibroblasts in vivo.

If the fibroblasts are not to be administered immediately, they can be incubated on ice at about 4° C. for up to 24-48 hours. The cells can be suspended in a physiological solution that has an appropriate osmolarity and has been tested for pyrogen and endotoxin levels. Such a solution typically does not contain phenol red pH indicator, and any serum preferably is the subject's serum rather than FBS or another xenogeneic serum. Fibroblasts can be suspended in, for example, Krebs-Ringer solution comprising 5% dextrose, or in any other physiological solution (e.g., physiological saline). The cells can be aspirated and administered to a subject in the incubation medium. The volume of saline or incubation medium in which the cells are suspended typically is related to such factors as the number of fibroblasts to be injected and the extent of the damage due to tissue degeneration or defect.

Any other suitable method also can be used to prepare compositions containing autologous, passaged fibroblasts. See, e.g., U.S. Pat. Nos. 5,858,390; 5,665,372; 5,660,850; and 5,591,444; as well as WO 99/60951, all of which are incorporated by reference in their entirety.

### 2. Compositions Containing Fibroblasts and Muscle Cells

Compositions of the invention can contain autologous, passaged fibroblasts together with passaged muscle cells. The muscle cells can be autologous or non-autologous (e.g., from another subject or from a cell line), although autologous muscle cells are particularly useful. The muscle cells can be striatal muscle cells or smooth muscle cells. Autologous striatal muscle cells can be isolated from, for example, a biopsy of muscle tissue from the head (e.g., the tongue, palatoglossus or temporalis muscle), neck, trunk (e.g., the sternocleidomastoid muscle), or limbs (e.g., the soleus or gastrocnemius muscle). Autologous muscle biopsies typically are obtained from sites that are easily accessible yet not highly visible from a cosmetic standpoint. Smooth muscle cells typically are non-autologous, and can be isolated from, for example, an aortic biopsy from an organ (e.g., heart) transplant donor. Non-autologous muscle cells also can be from muscle cell lines. Such cells are commercially available from, for example, ATCC and from Clonetics Corp. (San Diego, Calif.). Alternatively, muscle cells can be derived from stem cells isolated from, for example, a dermal biopsy. Any suitable method for culturing and differentiating stem cells can be used, including those methods known in the art.

To prepare a suspension of passaged muscle cells, autologous or non-autologous muscle tissue can be obtained from a muscle biopsy. Samples typically are about 0.5-1.0 cm³ in size, with a mass of about 0.5-1 g. The tissue can be dissociated by gentle agitation in culture medium containing, for example, the same antibiotics described for processing of biopsies to generate autologous fibroblast cultures. Any obvious connective or fatty tissue can be removed with a forceps. The remaining tissue sample can be minced into smaller pieces (e.g., pieces that are no larger than 1 mm³). Mincing with razor blades in trypsin is particularly useful. The minced suspension can be gently stirred in trypsin/EDTA to dissociate the cells, which then can be decanted into a fresh flask such that remaining tissue pieces are left behind. FBS can be added to neutralize the trypsin. The trypsinization steps can be repeated up to a maximum of three times, or until no pink tissue pieces remain, and the muscle cells can be collected by centrifugation. Muscle cells and/or muscle tissue (e.g., the biopsy sample) can be extensively washed in media containing antibiotics and antifungal agents, as described above for cultured fibroblasts.

Muscle cells can be cultured in any suitable medium. A 1:1 mixture of human muscle growth medium:conditioned medium (HuGM/CM) is particularly useful. HuGM typically contains Ham's F10, 10% FBS, 5% FBS (defined and supplemented with iron; Hyclone, Logan, Utah), 0.5% chick embryo extract (Gibco/Invitrogen, Carlsbad, Calif.), 100 U/ml penicillin, and 100 μg/ml streptomycin. CM is HuGM that has been conditioned by incubation with MRC-5 fibroblasts (available from, for example, ATCC). Other useful media that do not contain xenogeneic serum proteins (e.g., serum-free media) are those described above for autologous fibroblasts. The culture medium also can contain the same antibiotics described above for autologous fibroblasts.

Muscle cells can be plated in tissue culture plates or flasks of any suitable size (e.g., 96-well plates, 24-well plates, 12-well plates, or 35 mm, 60 mm or 100 mm dishes, or T-25, T-75, T-150, or T-500 flasks) and fed with fresh medium (e.g., 1:1 HuGM/CM) when they reach about 40% confluence. Cells then can be fed with growth medium (e.g., HuGM) every 2 to 3 days. When the muscle cells reach about 70 to 80% confluence, they can be trypsinized and seeded into fresh tissue culture dishes (e.g., 100 mm dishes) at about 5×10⁵-10×10⁵ cells per dish. Dishes typically are about 20% confluent after subculturing. Cells can be expanded and subcultured until a suitable number is obtained. Cells can be monitored for contamination by bacteria, fungus, yeast, or mycoplasma as described above. Once a suitable number of muscle cells is reached, they can be incubated in serum-free medium for 2-18 hours to remove immunogenic proteins (i.e., to render the cells substantially free of culture medium serum-derived proteins). Prior to administration to a subject, muscle cells typically are washed 2 to 4 times in serum-free medium or in PBS. PBS that does not contain Ca⁺² or Mg⁺² is particularly useful. Cells typically are washed by centrifugation and resuspension, and then are suspended for injection in an equal volume of isotonic solution that has an appropriate physiological osmolarity and is substantially free of pyrogens and foreign proteins. Isotonic saline is a particularly useful isotonic solution.

A pharmaceutically acceptable carrier can be added to the muscle cells before they are administered to a subject. Alternatively, if the cells are not to be administered immediately, they can be incubated on ice at about 4° C. for up to 24-48 hours. For such incubation, the cells can be suspended in a physiological solution that has an appropriate osmolarity and has been tested for pyrogen and endotoxin levels. Such a solution typically does not contain phenol red pH indicator, and any serum preferably is the subject's serum rather than FBS or another xenogeneic serum. Muscle cells can be suspended in, for example, Krebs-Ringer solution comprising 5% dextrose, or in any other physiological solution. The cells can be aspirated and administered to a subject in the incubation medium. The volume of saline or incubation medium in which the muscle cells are suspended typically is related to factors such as the number of cells to be injected and the extent of the damage due to tissue degeneration or defect.

Muscle cells can be frozen for future use. Cells can be trypsinized and resuspended in any suitable freezing medium (see above; e.g., medium containing 90% calf serum and 10% DMSO). Muscle cell suspensions then can be aliquoted into cryogenic freezing vials and frozen at about −70° C. to about −86° C. before transfer to liquid nitrogen, or they can be frozen in a Liquid Nitrogen Cryo-preservation Unit. Cells can be thawed and used to initiate secondary cultures for preparation of additional suspensions for later use in the same subject, thus avoiding the inconvenience of obtaining a second specimen.

The invention provides methods for making a composition for repairing or augmenting a tissue defect in a subject. These methods typically involve obtaining a dermal biopsy and preparing a suspension of autologous, passaged fibroblasts that are substantially free of immunogenic proteins (e.g., culture medium serum-derived proteins), obtaining a muscle biopsy and preparing a suspension of autologous, passaged muscle cells that also are free of these immunogenic proteins, and combining the fibroblasts with the muscle cells to generate a composition that can be used to treat a condition such as, for example, urinary incontinence, vesicoureteral reflux, or esophageal reflux. Alternatively, a suspension of non-autologous muscle cells can be prepared and combined with the fibroblasts.

### 3. Compositions Containing Biodegradable Acellular Matrix Components

Compositions of the invention that contain autologous, passaged fibroblasts with or without passaged muscle cells also can include biodegradable acellular matrix components. Such compositions are suitable for injection or implantation into a subject to repair tissue that has degenerated. The term “biodegradable” as used herein denotes a composition that is not biologically harmful and can be chemically degraded or decomposed by natural effectors (e.g., weather, soil bacteria, plants, animals). Examples of matrices that can be used in the present invention include, without limitation, acellular matrices comprising autologous and non-autologous proteins, and acellular matrices comprising biodegradable polymers.

Any of a number of biodegradable acellular matrices containing non-autologous proteins can be used in the compositions provided herein. Examples of biodegradable acellular matrices include matrices containing any type of collagen, or any type of collagen with glycosaminoglycans (GAG) cross-linked with, for example, glutaraldehyde. Other substances from which useful biodegradable acellular matrices can be made include hyaluron, hyaluronic acid, restalyn, and parleane. Matrices containing collagen include, without limitation, absorbable collagen sponges, collagen membranes, and bone spongiosa. Useful types of collagen include, for example, bovine collagen (e.g., Zyderm® and Zyplast®, commercially available from McGhan Medical Corporation, Santa Barbara, Calif.), porcine collagen, human cadaver collagen (e.g., Fascian™ (Fascia Biosystems, LLC, Beverly Hills, Calif.), Cymetra (LifeCell Corp., Branchburg, N.J.), or Dermalogen™, formerly produced by the Collagenesis Corp.), and autologous human collagen (Autologen®, see below). Fascian™ is particularly useful. This product is available in five different particle sizes, any of which can be used in compositions and methods described herein. Particles that are 0.25 mm in size are particularly useful.

Absorbable collagen sponges can be purchased from, for example, Sulzer Calcitek, Inc. (Carlsbad, Calif.). These collagen sponge dressings, sold under the names CollaTape®, CollaCote®, and CollaPlug®, are made from cross-linked collagen extracted from bovine deep flexor (Achilles) tendon, and GAG. These products are soft, pliable, nonfriable, and non-pyrogenic. Greater than 90% of a collagen sponge typically consists of open pores.

Biodegradable acellular matrices can contain collagen (e.g., bovine or porcine collagen type I) formed into, for example, a thin membrane. One such membrane is manufactured by Sulzer Calcitek and is marketed as BioMend™. Another such membranous matrix is marketed as Bio-Gide® by Geistlich Söhne AG (Wolhusen, Switzerland), and is made of porcine type I and type III collagen. Bio-Gide® has a bilayer structure, with one surface that is porous and allows the ingrowth of cells, and a second surface that is dense and prevents the ingrowth of fibrous tissue.

Biodegradable acellular matrices also can be made from bone spongiosa formed into granules or blocks. This material consists of animal (e.g., human, non-human primate, bovine, sheep, pig, or goat) bone from which substantially all organic material (e.g., proteins, lipids, nucleic acids, carbohydrates, and small organic molecules such as vitamins and non-protein hormones) has been removed. This type of matrix is referred to herein as an “anorganic matrix”. One such matrix, which is marketed as Bio-Oss® spongiosa granules and Bio-Oss® blocks, is manufactured by Geistlich Söhne AGC. This company also manufactures a block-type matrix (Bio-Oss® collagen) that contains anorganic bone and additionally contains approximately 10% collagen fibers by weight.

Other useful biodegradable acellular matrices can contain gelatin, polyglycolic acid, cat gut, demineralized bone, anorganic bone, or hydroxyapatite, or mixtures of these substances. A matrix made from demineralized human bone, for example, is formed into small blocks and marketed as DynaGraft® by GenSci Regeneration Laboratories, Inc. (Toronto, Ontario). Demineralized bone can be combined with, for example, collagen to produce a matrix in the form of a sponge, block, or membrane. Synthetic polymers made from one or more monomers also can be used to make biodegradable acellular matrices that are useful herein. The matrices can be made from one or more of such synthetic polymers. The synthetic polymers also can be combined with any of the above-mentioned substances to form matrices. Different polymers forming a single matrix can be in separate compartments or layers. For example, W. L. Gore & Associates, Inc. (Flagstaff, Ariz.) manufactures a porous biodegradable acellular matrix (GORE RESOLUT XT Regenerative Material). This matrix is composed of a synthetic bioabsorbable glycolide and trimethylene carbonate copolymer fiber into which cells can migrate, attached to an occlusive membrane that is composed of a synthetic bioabsorbable glycolide and lactide copolymer that does not permit ingrowth of cells.

After a biodegradable acellular matrix has been selected, a concentrated suspension of autologous passaged fibroblasts with or without passaged muscle cells can be evenly distributed on the surface of the matrix. A concentrated suspension typically is used in order to avoid exceeding the capacity of the matrix to absorb the liquid suspension. For example, a cell suspension applied to a GORE RESOLUT XT matrix generally can have a volume between about 94 μl and about 125 μl and contain between about 2.0×10⁶ cells and about 4.0×10⁶ cells per square centimeter of matrix. Cells can be allowed to attach to the matrix without further addition of media. Incubation of the cells with the matrix can occur at, for example, about 37° C. for about 1-2 hours. Cells typically are attached to and evenly distributed throughout the matrix material after about sixty minutes of incubation. At this time, the culture vessels containing the cell-loaded matrices can be supplemented with additional growth medium, and cells can be cultured in the matrix for about 3 to 4 days. Because the cells are added to the matrix at high density so as to substantially fill the space within the matrix, little or no proliferation occurs during the 3-4 day culture period. Indeed, significant cell proliferation typically is undesirable during this period because dividing fibroblasts can secrete enzymes (e.g., collagenase) that can degrade or partially degrade the matrices.

The matrix with the cells typically is washed (e.g., at least 3 washes of 10 minutes each) with, for example, saline or medium that is free of serum and phenol red, in order to substantially remove immunogenic proteins (e.g., culture medium serum-derived proteins if medium containing non-autologous serum was used for the matrix seeding step) that could elicit an immune response when administered to a subject. Fresh PBS can be used for each wash. The matrix then can be incubated (e.g., 2 hour-long incubations) in fresh PBS prior to use. After incubation, the matrix containing autologous, passaged fibroblasts with or without passaged muscle cells can be placed at the area of tissue degeneration or defect.

For collagen sponge matrices (e.g. CollaCote®), approximately 1.5×10⁷ to 2.0×10⁷ cells in approximately 1.5 ml of growth medium can be seeded onto a 2 cm by 4 cm thin (approximately 2.5 to 3.0 mm in thickness) sponge. The sponge then can be incubated at 37° C. for about 1-2 hours without further addition of medium to allow substantially all the fibroblasts to adhere to the matrix material. After cell adherence, additional growth medium can be added to the matrix and cell composition, which then can be incubated at 37° C. for 3-4 days with a daily change of medium. If medium containing non-autologous serum was used for the cell seeding step, the composition can be removed from growth medium containing such serum and washed repeatedly (e.g., 3 times or more) with PBS. After each addition of PBS, the matrix can be incubated for 10-20 minutes prior to discarding the PBS. After the final wash, the composition can either be administered immediately to a subject, or can be transferred to a shipping vial containing a physiological solution (e.g., Kreb's Ringer solution) and incubated at about 4° C. for up to about 24-48 hours.

For a membranous matrix (e.g. BioMend™), approximately 3×10⁶ to 8×10⁶ fibroblasts in about 100 μl of growth medium can be seeded onto a 15 mm×20 mm thin (approximately 0.5 to 1.0 mm in thickness) membrane. The membrane can be incubated at 37° C. for about 30-60 minutes without further addition of medium to allow substantially all of the cells to adhere to the matrix material. After cell adherence, additional growth medium can be added to the matrix and cell composition, which then can be incubated at 37° C. for 2-3 days with a daily change of medium. The cells typically are added to the matrix at high density (see above) so as to substantially fill the space within the matrix available for cells. Washing of the composition and either immediate use or incubation can be as described above for the sponge matrices.

In the case of a block matrix such as the above described anorganic matrix (e.g., the Bio-Oss® block) or a demineralized bone matrix (e.g., the Dynagraft™ matrix), approximately 1.2×10⁷ to 2.0×10⁷ cells in approximately 100 to 150 μl of growth medium can be seeded into a 1 cm×1 cm×2 cm cubic block of matrix material. Cells typically are seeded slowly onto one face of the block face. Once the medium and cells have been absorbed into the block, another face of the block can be seeded in a similar fashion. The procedure can be repeated until all faces of the block have been seeded and the block is fully saturated with medium. Care should be taken to avoid adding excess medium and thereby causing leakage of medium and cells from the block. The composition then can be incubated at 37° C. for about 60-120 minutes without further addition of medium to allow substantially all the cells to adhere to the matrix material. After cell adherence, additional growth medium can be added to the matrix and cell composition, which then can be incubated at 37° C. for 2-3 days with a daily change of medium. The cells typically are added to the matrix at high density (see above) so as to substantially fill the space within the matrix available for cells with the same result described above. Washing of the composition and either immediate use or incubation are as described above for the sponge matrices.

Compositions containing autologous, passaged fibroblasts and a small particle biodegradable matrix (e.g., Fascian™, Cymetra™, or Dermalogen™) can be prepared by mixing the components by, for example, passing them back and forth between two syringes that are connected via a luer lock. Fascian™, for example, is typically available in syringes (e.g., 3 cc syringes) at 80 mg/syringe. Fascian™ particles can be washed directly in the syringe prior to use by taking up a small volume (e.g., 1.5 ml) of a wash buffer (e.g., isotonic saline or Kreb's Ringers solution containing dextrose) into the syringe, connecting the first syringe to a second syringe via a luer lock, and passing the particles and wash solution back and forth between the two syringes several times. To separate the particles from the wash solution, the mixture can be transferred to a sterile tube and the Fascian™ particles allowed to settle. The solution can be removed (e.g., decanted or aspirated), and the washing process can be repeated as desired by taking up the particles into a fresh syringe (e.g., through an 18 gauge or 20 gauge needle).

When the filler particles are suitably washed, they can be mixed with fibroblasts and, optionally, muscle cells using the same procedure as for washing. Cells (e.g., 1×10⁷ to 3×10⁷ cells) can be suspended in solution (e.g., 1.5 ml of Kreb's Ringers solution with 5% dextrose) and taken up into a syringe. The syringe containing the cells can be connected to a syringe containing the filler particles via a luer lock, and the two components can be mixed by passing them back and forth between the syringes. The mixture then can be transferred to a T-25 tissue culture flask or to a tissue culture dish or a tube so that the cells can attach to the filler particles. Alternatively, the mixture can remain in the syringes while attachment occurs, although this may be more detrimental to the cells. The mixture can be incubated overnight and then transferred to a container (e.g., a vial or a tube) for delivery to a clinician, or transferred to a syringe for administration to a subject. A container to be delivered to a clinician can be kept on ice during delivery. When such small particle acellular biodegradable matrices are used, a suspension of the cell-containing particles can optionally be injected rather than implanted into an area of tissue degeneration or defect.

When cultured muscle cells are included in a composition containing fibroblasts and a biodegradeable acellular matrix, they can be mixed with the fibroblasts prior to seeding into the matrices. Alternatively, they can be seeded into the matrices prior to or after seeding of the fibroblasts. When the muscle cells are seeded before or after the fibroblasts, the second seeding can be performed immediately after the first seeding or after the cells of the first seeding have substantially adhered to the matrix material.

The invention also provides methods for making compositions that contain autologous, passaged fibroblasts together with matrix components. These methods typically involve providing a suspension of autologous, passaged fibroblasts that are substantially free of immunogenic proteins (e.g., culture medium serum-derived proteins), providing a biodegradable acellular matrix, incubating the biodegradable acellular matrix with the fibroblast suspension such that the fibroblasts integrate on and within the matrix, thus forming a composition for repairing or augmenting tissue. These methods also can include adding a suspension of passaged muscle cells (e.g., autlogous, passaged muscle cells) to the matrix, either together or separately from the fibroblasts.

### 4. Compositions Containing Bulking Agents

Compositions of the invention can contain autologous, passaged fibroblasts together with one or more biodegradable acellular injectable filler materials (i.e., bulking agents). The compositions are suitable for injection into a subject in order to repair tissue that has degenerated. In addition to fibroblasts and fillers, the compositions also can contain passaged muscle cells (typically autologous muscle cells; see above). In an injectable composition that contains autologous, passaged fibroblasts with or without muscle cells, together with a biodegradable, acellular filler, the cells typically are mixed with the filler in a ratio of approximately 1:1 by volume.

Numerous types of biodegradable, acellular injectable fillers can be added to compositions of the invention. A filler can consist of autologous proteins, including any type of collagen obtained from a subject. An example of such a filler is Autologen®, formerly produced by Collagenesis Corp. (Beverly, Mass.). Autologen® is a dispersion of autologous dermal collagen fibers from a subject, and therefore should not elicit an immune response when readministered to the subject with muscle cells and/or fibroblasts. In order to obtain Autologen®, a specimen of tissue (e.g., dermis, placenta, or umbilical cord) is obtained from a subject and forwarded to Collagenesis Corp., where it is processed into a collagen-rich dispersion. Approximately one and a half square inches of dermal tissue can yield one cubic centimeter (cc) of Autologen®. The concentration of Autologen® can be adjusted depending upon the amount required to correct defects or augment tissue within the subject. The concentration of Autologen® in the dispersion can be, for example, at least about 25 mg/L (e.g., at least 30 mg/L, at least 40 mg/L, at least 50 mg/L, or at least 100 mg/L).

An acellular injectable filler material also can contain non-autologous proteins, including any type of collagen. Numerous collagen products are commercially available and can be used in compositions of the invention. Human collagen products also are commercially available. Examples of commercially available collagen include, without limitation, reconstituted bovine collagen products such as Zyderm® and Zyplast®, which contain reconstituted bovine collagen fibers that are cross-linked with glutaraldehyde and suspended in phosphate buffered physiological saline with 0.3% lidocaine. These products are produced by McGhan Medical Corporation of Santa Barbara, Calif. Porcine collagen products also are commercially available.

Other examples of useful filler materials include, but are not limited to, solubilized gelatin, polyglycolic acid, or cat gut sutures. A particular gelatin matrix implant, for example, is sold under the mark Fibril®. This filler contains equal volumes of (1) a mixture of porcine gelatin powder and o-aminocaproic acid dispersed in a 0.9% (by volume) sodium chloride solution, and (2) an aliquot of plasma from the subject. Other substances useful as fillers include hyaluron, hyaluronic acid, restalyn, and parleane.

The invention also provides methods for making compositions that contain autologous, passaged fibroblasts and biodegradable acellular fillers, with or without passaged muscle cells. These methods typically involve providing a suspension of autologous, passaged fibroblasts and, optionally, muscle cells that are substantially free of immunogenic proteins (e.g., culture medium serum-derived proteins), providing one or more biodegradable acellular filler materials, and combining the filler with the fibroblast and muscle cell suspension. Alternatively, separate suspensions of the two cell types can be combined with the filler.

### 5. Device for Administering Compositions of the Invention

The invention also provides a device for delivering compositions containing autologous, passaged cells to a point proximate to the site of tissue degeneration or defect (e.g., the urethra, the ureteral orifice, or the lower esophageal sphincter). Such a device can consist of a sterile hypodermic syringe having a syringe chamber, a piston disposed therein, an orifice communicating with the chamber, and a pharmaceutical composition that contains autologous, passaged fibroblasts, such that the pharmaceutical composition is disposed within the chamber. The pharmaceutical composition can contain autologous, passaged fibroblasts and one or more of the following: passaged muscle cells (e.g., autologous, passaged muscle cells), a pharmaceutically acceptable carrier, a biodegradable acellular filler, and biodegradable acellular matrix components. The hypodermic syringe can have a capacity of any suitable size (e.g., 1 cc, 3 cc, 10 cc, or more than 10 cc). The syringe also can be connected to a needle of an appropriate size (e.g., 14 gauge, 16 gauge, 18 gauge, 20 gauge, 23 gauge, 25 gauge, 27 gauge, or 30 gauge) and length (e.g.,less than 20 cm, 20 cm, 25 cm, 30 cm, 35 cm, or 40 cm, or more than 40 cm).

### 6. Methods for Repairing or Augmenting Tissue

Methods of the invention can be used to administer an effective amount of a composition of the invention to a subject so as to repair or augment tissue within the subject. As used herein, the term “effective amount” refers to an amount of a pharmaceutical composition that can provide suitable bulk to correct a tissue defect in the subject, or that can promote tissue regeneration in tissue that has degenerated in a subject. Methods of the invention are particularly useful for administering pharmaceutical compositions to treat tissue degeneration or defects that are associated with disorders such as, for example, urinary incontinence, vesicoureteral reflux, or GERD.

Methods of the invention typically involve administering one or more compositions of the invention to a subject by, for example, injection or implantation. When a combination of autologous, passaged fibroblasts, passaged muscle cells (e.g., autologous, passaged muscle cells), and filler is to be administered to a subject, the components can be administered simultaneously or separately. For example, autologous, passaged fibroblasts can be administered to a subject as one injection, autologous, passaged muscle cells can be administered as a separate injection, and a filler material can be administered as yet another injection. The injections can be separated by any suitable length of time (e.g., 5 minutes, 30 minutes, 1 day, 3 days, 1 week, 2 weeks, or more than 2 weeks). Alternatively, the components can be combined prior to injection. For example, a composition containing autologous, passaged fibroblasts, passaged muscle cells, and a filler can be administered in a single injection. Alternatively, a mixture of autologous, passaged fibroblasts and passaged muscle cells can be administered as one injection, and a filler can be administered in a separate injection.

Methods of the invention can be used to treat any mammalian species (e.g., humans, non-humans, primates, dogs, cows, pigs, horses, sheep, cats, rabbits, mice, rats, guinea pigs, hamsters, or gerbils). Methods of the invention are particularly useful for treating humans.

Methods of the invention can be used to treat urinary incontinence and/or vesicoureteral reflux by reforming or repairing tissue (e.g., sphincter structures) surrounding the urethra, ureters, and esophagus, thus causing a reduction in size of abnormally wide and loose lumens. These methods involve placement (e.g., injection or implantation) of compositions of the invention into the regions surrounding the urethra, ureters, or esophagus, or directly into a pocket created in the region to be repaired or augmented.

The male urethra is divided into the prostatic, membranous, and penile regions. The membranous region is the thickest portion of the urethra, and passes through the genitourinary diaphragm. The skeletal muscle layer of the membranous urethra contains the external (or voluntary) urinary sphincter, which forms almost a complete ring around the urethra. Methods of the invention can be used to administer (e.g., by injection or implantation into the urethral wall) compositions containing autologous fibroblasts to a subject with urinary incontinence to improve the function of a damaged or defective membranous urethra.

The female urethra is a very short and dilatable tubular structure measuring about 4 cm in length. The urethra begins at the bladder outlet and extends through the perineal membrane, running behind the pubic symphysis and ending at the external urethral orifice in the perineum. The female urethra represents the entire sphincter mechanism for the bladder. Internally it is covered by a mucous layer and its core is a strong muscular wall composed of three main muscular layers. The middle layer contains condensed striated muscle fibers that form a ring; incontinence can result if these muscle fibers are partially deficient. Urethral function can be altered or damaged by anatomical problems within the urethra or within adjacent organs such as the vagina and the bladder. The function of a damaged or defective membranous urethra can be improved using methods of the invention to administer (e.g., by injection or implantation into the urethral wall) compositions containing autologous fibroblasts and, optionally, muscle cells (e.g., autologous muscle cells). Such compositions can contain the cells in a diluent (e.g., saline), in a suspension containing a biodegradable filler material, or seeded into a biodegradable acellular matrix.

The ureter is a muscular conduit that contracts in response to the stretch reflex during transport of urine from the kidney to the bladder. The orifice of the distal ureter into the bladder is known as the ureteral meatus, and is located in the posterolateral aspect of the bladder wall at the sides of an underlying muscle and a triangular structure called the vesical trigone. The musculature of the ureter and the vesical trigone is in continuity because the ureteral muscular coat passes through the meatus and fans out on the floor of the bladder. The length of the intravesical ureter and the intrinsic longitudinal muscular coat of the submucosal ureter that inserts into the superficial trigone are critical to the normal function of the distal ureter. These factors are important for the shape of the ureteral orifice, and when the orifice has an altered shape there is an increased tendency for malposition of the orifice, shorter portions of intravesical ureter, and reflux as a consequence.

Procedures to improve the function of the urethral sphincter or ureteral musculature can, optionally, be conducted under local or general anesthesia. For injection into ureteral structures, a cystoscopy can be performed, typically on an outpatient basis or even during an office visit. A cystoscope can be introduced into the urethra such that its tip is located at a proper visual distance from the abnormal distended urethra/ureter lumen. Injections can be done either periurethrally or transurethrally.

To treat a male with urinary incontinence, a needle of any suitable gauge and length (e.g., a 20 gauge×35 cm needle) can be introduced through the working channel of the cystoscope, oriented into the urethral surrounding tissue from the distended lumen to the outside, and advanced into the tissue. A composition of the invention that contains autologous fibroblasts, with or without muscle cells (e.g., autologous muscle cells) and, optionally, a biodegradable acellular filler or matrix, can be injected into the urethral surrounding tissue until the desired narrowing of the lumen is achieved.

To treat a female with urinary incontinence, a needle of any suitable gauge and length (e.g., a 20 gauge×30 cm needle) can be inserted periurethrally, such that the needle is directed with the bevel downward and is advanced into the bladder neck with the direction of the needle placement guided by the axis of the cystoscope. Observation of the ideal needle placement into the surrounding mucosal tissue can be obtained by gentle movement of the needle observed from the cystoscopic visual field before injecting the cell preparation packed into a syringe connected to the needle. Injections can be made at the “3 o'clock and/or 9 o'clock positions”. The narrowing of the lumen can be continually monitored by the cystoscope until the injection is complete.

To treat vesicoureteral reflux, a subject can be placed in a dorsal or modified lithotomy position and a cystoscope can be inserted and advanced until the ureters are visualized. A needle (e.g., an 18 gauge needle, a 20 gauge needle, a 23 gauge needle, a 25 gauge needle, or a 27 gauge needle) that is about 25 to 40 cm in length (e.g., 25 cm, 30 cm, 35 cm, or 40 cm in length) can be advanced through the working channel. The needle tip can be inserted into the bladder mucosa under direct vision at a “6 o'clock position” into the subureteral space, approximately 4 to 6 mm distal to the ureteral orifice. Proper placement of the needle may be facilitated by placing a French catheter into the ureter. The needle then can be advanced proximally. A composition containing autologous fibroblasts with or without muscle cells (e.g., autologous muscle cells) and optionally with a biodegradable acellular filler or matrix, can be injected slowly until a bulge nearly obliterates the ureteral orifice. To prevent extravasion, a single precise injection typically is performed, and the needle can be kept in position for 2 to 3 minutes before withdrawal.

The esophagus is a muscular canal that is about 8 inches in length and extends from the pharynx to the stomach. The upper and lower ends of the esophagus both have sphincter structures that remain closed except during swallowing. A deficiency in the lower esophageal sphincter (LES) can allow stomach contents to back up into the esophagus, thus causing GERD.

To improve the function of the LES and alleviate symptoms of GERD, compositions of the invention can be placed (e.g., injected or implanted) into the esophagus at or near the LES. An endoscope can be used to visualize the LES, and a needle of any suitable size and length (e.g., a 23 gauge×25 cm needle, or a 23 gauge×1.5 cm needle attached to a cathether) is inserted through the working channel of the endoscope. With the bevel facing inward toward the esophageal lumen, the needle then can be inserted into the esophageal mucosa slightly proximal to the LES. A composition of the invention can be injected into the wall of the esophagus at several locations (e.g., at the 3 o'clock, 6 o'clock, 9 o'clock, and 12 o'clock positions), until the desired bulking of the mucosa is achieved.

The invention also provides methods for administering compositions of the invention for augmentation and/or repair of dermal, subcutaneous, and fascial tissues. Compositions containing autologous, passaged fibroblasts, with or without passaged muscle cells, matrix components, and/or fillers can be injected or implanted into a subject to treat, for example, scarring, cellulite, skin laxness or skin thinning, wrinkles, wounds (e.g., acute, chronic, partial or full-thickness wounds, bums, pressure sores, and ulcers), breast deficiencies, periodontal disorders, defects of an oral mucosa, trauma to an oral mucosa, diabetes, venous stasis, hernias, damage to ligaments, tendons and muscles of the joints, and allopecia. Methods for treating these conditions can involve, for example, injecting into the site of the deficiency or defect a composition that contains autologous, passaged fibroblasts and passaged muscle cells (e.g., autologous, passaged muscle cells), wherein the cells are substantially free of culture medium serum-derived proteins.

The invention will be further described in the following examples, which do not limit the scope of the invention described in the claims.

### EXAMPLES

### Example 1

### Method of Obtaining an Injectable Fibroblast Suspension

Dermal fibroblast cultures were initiated from a 1-5 mm full thickness biopsy specimen of the skin, or a thicker specimen if the tissue was available. Because of the phenomenon of allograft rejection, it was essential that the cultured fibroblasts be histcompatible with the host. Histocompatability was ensured by obtaining a biopsy from the subject to be treated, and culturing the fibroblasts from this specimen.

Before initiation of the culture, the biopsy was washed repeatedly with antibiotic and antifungal agents (see above). The epidernis and the subcutaneous adipocyte-containing tissue were removed so that the resultant culture was substantially free of non-fibroblast cells. The remaining specimen of dermis was finely divided with a scalpel or scissors. Individual pieces of tissue were placed with a forceps onto the dry surface of a T-25 tissue culture flask and allowed to attach for between 2 and 5 minutes, or as needed depending on the tissue and the temperature in the laboratory and the culture hood. A small amount of medium (generally 1.5 to 2 ml) was slowly and carefully added so as not to displace the attached tissue fragments. After 48-72 hours of incubation, the flask was fed with additional medium containing standard antibiotics. Cells were maintained in medium with antibiotics for 14 days, from which point the cells were incubated in antibiotic-free medium containing Gentamicin.

During the early stages of culture it was desired that the tissue fragments remain attached to the bottom of the culture flasks; fragments that detached were reimplanted into new flasks. The fibroblasts were stimulated to grow by a brief exposure to trypsin/EDTA. This exposure was too brief to release the cells from their attachment. This process allowed a more even distribution of the cells over the flask surface, and promoted more rapid proliferation toward confluence. Immediately after the cultures became established and were approaching confluence, samples of the fibroblasts were removed for frozen storage. Because the number of passages of human fibroblasts typically is limited, the storage of early passage fibroblasts was preferred. Freezing medium typically contained 7-10% DMSO and 20% autologous serum or FBS, although cells also could be frozen in glycerol or 90% serum.

Once the cells were confluent, they were passaged into new flasks by trypsinization. For expansion, individual flasks were split 1:3 into triple bottom T-150 flasks that had a total culture area of 450 cm². These flasks were seeded with about 6×10⁶ cells and yielded about 1.8×10⁷ cells. When the capacity of the flasks was reached (typically after 7-10 days of culture) the growth medium was replaced with serum-free complete medium. Thereafter the cells were incubated at 37° C. for at least 2 hours (typically more than 12 hours) in protein-free medium. The incubation in serum-free medium substantially removed from the cells the proteins that were derived from the fetal bovine serum which, if present, would be immunogenic in the subject and would cause an allergic reaction.

At the end of the incubation in serum-free medium, the cells were removed from the tissue culture flasks by trypsin/EDTA, washed extensively by centrifugation and resuspension, and suspended for injection in an equal volume of injectable isotonic saline. Five to ten triple bottom T-150 flasks, grown to capacity, yielded about 3×10⁷ to 1×10⁸ cells, which was sufficient to make up about 1-3 ml of suspension.

Alternatively, the cells could be transported at 4° C. as long as they were injected within 18 hours of the time that the suspension was made. Cells were suspended in an equal volume of complete medium, except for the absence of phenol red pH indicator and the replacement of FBS with serum from the subject. The cells were aspirated and injected in the transport medium. The volume of saline or transport medium in which cells were suspended was not critical.

### Example 2

### Method of Obtaining an Injectable Cell Population in a Viscous Suspension

When a large volume of bulk-enhancing material was required, an alternative method of preparing an injectable suspension of cells was used. This method was identical to the methods described in Example 1 until a population of about 10⁶ cells was obtained. A plasma clot was then formed in the bottom of a 60 mm or 100 mm tissue culture dish by adding 1 ml of the subject's plasma and 50-100 μl of 300 mM CaCl₂. Cultured dermal fibroblasts (10⁶ cells in 2-10 ml) were seeded on the surface of the clot and cultured for a further 7 days in complete medium. After 7 days, the complete medium was exchanged for serum-free medium. An hour after the initial replacement of medium, the medium was again removed and replaced with fresh serum-free medium. Cells were incubated for another 14-18 hours. The clot was then aspirated into a syringe and injected as needed.

### Example 3

### Method of Obtaining an Injectable Suspension of Muscle Cells

A muscle biopsy is performed to collect a sample that is about 0.5-1.0 cm³ in size, such that about 0.5-1 g of tissue is obtained. The tissue is dissociated by gentle agitation in Ham's F10 medium, and any obvious connective or fatty tissue is removed with a forceps. The remaining tissue sample is transferred to 5 ml of trypsin in a 100 mm dish, and sterile razor blades are used to mince the tissue into pieces no larger than 1 mm³. The minced suspension is then transferred to a sterile flask containing a stir bar, and trypsin/EDTA is added to a final volume of 20-25 ml. The suspension is very gently stirred for 20 minutes at 37° C. Once the tissue pieces settle to the bottom of the flask, the supernatant is decanted to a 50 ml plastic centrifuge tube on ice. FBS is added to a final concentration of 10% in order to neutralize the trypsin. The trypsinization steps are repeated up to a maximum of three times, or until no pink tissue pieces remain. The supernatants are centrifuged at 800-900 g for 5 minutes.

The cell pellets are pooled in 10 ml of 1:1 Human muscle growth medium/conditioned media (HuGM/CM). HuGM contains Ham's F10, 10% FBS, 5% bovine calf serum (defined and supplemented with iron; Hyclone, Logan, Utah), 0.5% chick embryo extract (Gibco/Invitrogen, Carlsbad, Calif.), 100 U/ml penicillin, 100 μg/ml streptomycin. CM is HuGM that is conditioned by incubation with MRC-5 fibroblasts (American Type Culture Collection, Manassas, Va.) overnight and then filtered through 0.45 μm filters. Each 0.1 g of tissue typically yields 5×10³ cells.

Cells are plated in tissue culture plates of any suitable size (e.g., 35 mm, 60 mm, or 100 mm) and fed with 1:1 HuGM/CM at day 1 or 2 if the cells are 30-40% confluent. Otherwise, cells are fed with 1:1 HuGM/CM at day 4 or 5 or when cells reach 40% confluence (whichever is sooner). Cells then are fed with HuGM every 2-3 days, unless they are less than 40% confluent, in which case they are fed with 1:1 HuGM/CM. When cells reach 70-80% confluence, they are subjected to trypsinization, dispersed in HuGM, and seeded into fresh 100 mm dishes at 5-10×10⁵ cells per dish in 10 ml of HuGM. Dishes typically are about 20% confluent after subculturing. Cells are expanded and subcultured until a suitable number is obtained.

Muscle cells are frozen for future use. Cells are trypsinized and collected into a 15 ml tube. The number of cells is determined using a hemocytometer, and the cells are centrifuged at 800-900 g for 2 minutes. The medium is aspirated and cells are resuspended in freezing medium (90% calf serum, 10% dimethylsulfoxide) to give approximately 2×10⁶ cells/ml. The cell suspension is aliquoted at 0.5 ml per 2 ml cryogenic freezing vial, and vials are frozen in a foam-filled box at −70° C. before transfer to liquid nitrogen.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition for repairing tissue that has degenerated in a subject as a result of a disease, disorder, or defect in said subject, wherein said composition comprises autologous, passaged fibroblasts and autologous, passaged muscle cells, wherein said composition is substantially free of culture medium serum-derived proteins

2. The composition of claim 1, wherein said disease, disorder, or defect is associated with urinary incontinence, vesicoureteral reflux, or gastroesophageal reflux.

3. The composition of claim 1, wherein said autologous fibroblasts are from gums, palate, skin, lamina propria, connective tissue, bone marrow, or adipose tissue of said subject.

4. The composition of claim 1, wherein said autologous muscle cells are striatal muscle cells

5. The composition of claim 4, wherein said striatal muscle cells are from the tongue, palatoglossus, temporalis muscle, soleus, gastrocnemius, or stemocleidomastoid muscle of said subject

6. The composition of claim 1, wherein said autologous muscle cells are smooth muscle cells.

7. The composition of claim 1, wherein said composition further comprises a biodegradable acellular matrix, wherein said fibroblasts and muscle cells are integrated within and on said matrix.

8. The composition of claim 7, wherein said matrix, prior to combination with said fibroblasts and muscle cells, comprises one or more substances selected from the group consisting of collagen, glycosaminoglycans, gelatin, polyglycolic acid, cat gut, demineralized bone, hydroxyapatite, and anorganic bone.

9. The composition of claim 8, wherein said one or more substances comprise collagen and glycosaminoglycans, cross-linked with glutaraldehyde.

10. The composition of claim 8, wherein said one or more substances is collagen

11. The composition of claim 10, wherein said collagen is bovine collagen.

12. The composition of claim 10, wherein said collagen is porcine collagen type I or porcine collagen type III.

13. The composition of claim 8, wherein said one or more substances are selected from the group consisting of gelatin, polyglycolic acid, cat gut, demineralized bone, and hydroxyapatite.

14. The composition of claim 13, wherein said one or more substances are selected from the group consisting of gelatin, polyglycolic acid, and cat gut.

15. The composition of claim 7, wherein sufficient fibroblasts and muscle cells integrate on and within said matrix to substantially fill the space on and within said matrix available for cells.

16. A method for making a composition for repairing tissue that has degenerated in a subject as a result of a disease, disorder, or defect in said subject, said method comprising:
(a) providing a biopsy of fibroblast-containing tissue from said subject;
(b) separating autologous fibroblasts from said biopsy;
(c) culturing said autologous fibroblasts under conditions that produce autologous fibroblasts that are substantially free of culture medium serum-derived proteins;
(d) exposing said cultured autologous fibroblasts to conditions that result in suspension of said fibroblasts;
(e) providing a biopsy of muscle tissue from said subject;
(f) culturing autologous muscle cells isolated from said muscle tissue under conditions that result in muscle cells that are substantially free of culture medium serum-derived proteins;
(g) exposing said cultured autologous muscle cells to conditions that result in suspension of said muscle cells; and
(h) combining said fibroblasts with said muscle cells.

17. The method of claim 16, wherein said disease, disorder, or defect is associated with urinary incontinence, vesicoureteral reflux, or gastroesophageal reflux.

18. The method of claim 16, wherein the fibroblast-containing tissue is selected from the group consisting of gums, palate, skin, lamina propria, connective tissue, bone marrow, and adipose tissue.

19. The method of claim 16, wherein the muscle tissue is selected from the group consisting of tongue, palatoglossus, temporalis muscle, soleus, gastrocnemius, and stemocleidomastoid muscle.

20. The method of claim 16, wherein said culturing of said fibroblasts or said muscle cells comprises: (1) incubation in a culture medium comprising between 0.1% and about 20% human or non-human serum, followed by (2) incubation in a serum-free culture medium.

21. The method of claim 16, wherein said culturing of said fibroblasts or said muscle cells comprises incubation in serum-free medium.

22. The method of claim 16, wherein said culturing of said fibroblasts or said muscle cells is in a medium comprising one or more reagents that prevents the growth of mycoplasma.

23. The method of claim 22, wherein said one or more reagents comprise tylosin.

24. The method of claim 23, wherein said one or more reagents further comprises one or more compounds selected from the group consisting of gentamicin, ciprofloxacine, alatrofloxacine, azithromycin, and tetracycline.

25. The method of claim 16, wherein said conditions that result in suspension of said fibroblasts or said muscle cells comprise a proteolytic enzyme.

26. A method for making a composition for repairing tissue that has degenerated in a subject as a result of a disease, disorder, or defect in said subject, wherein said method comprises:
(a) providing autologous, passaged fibroblasts and autologous, passaged muscle cells
(b) providing a biodegradable acellular matrix; and
(c) incubating said fibroblasts and muscle cells with said biodegradable acellular matrix such that said fibroblasts and muscle cells integrate on and within said biodegradable acellular matrix, wherein said incubation results in a composition for repairing tissue, and wherein the conditions of said incubation are such that said composition is substantially free of culture medium serum-derived proteins.

27. The method of claim 26, wherein said disease, disorder, or defect is associated with urinary incontinence, vesicoureteral reflux, or gastroesophageal reflux.

28. The method of claim 26, wherein the step of providing autologous, passaged fibroblasts and autologous, passaged muscle cells comprises:
(a) providing a biopsy of fibroblast-containing tissue from said subject;
(b) separating autologous fibroblasts from said biopsy;
(c) culturing said fibroblasts;
(d) suspending said fibroblasts;
(e) providing a biopsy of muscle tissue from said subject;
(f) isolating muscle cells from said muscle tissue;
(g) culturing said muscle cells; and
(h) suspending said muscle cells.

29. The method of claim 28, wherein said fibroblast-containing tissue is selected from the group consisting of gums, palate, skin, lamina propria, connective tissue, bone marrow, and adipose tissue.

30. The method of claim 28, wherein said muscle tissue is selected from the group consisting of tongue, palatoglossus, temporalis muscle, soleus, gastrocnemius, and stemocleidomastoid muscle.

31. The method of claim 28, wherein said culturing of said fibroblasts and said muscle cells is in a medium comprising a reagent that prevents the growth of mycoplasma.

32. The method of claim 31, wherein said reagent comprises tylosin.

33. The method of claim 32, wherein said reagent further comprises one or more compounds selected from the group consisting of gentamicin, ciprofloxacine, alatrofloxacine, azithromycin, and tetracycline.

34. The method of claim 26, wherein said biodegradable acellular matrix, prior to combination with said suspensions of said fibroblasts and said muscle cells, comprises one or more substances selected from the group consisting of collagen, glycosaminoglycans, gelatin, polyglycolic acid, cat gut, demineralized bone, hydroxyapatite, and anorganic bone.

35. The method of claim 34, wherein said one or more substances comprise collagen and glycosaminoglycans, cross-linked with glutaraldehyde.

36. The method of claim 34, wherein said one or more substances are selected from the group consisting of gelatin, polyglycolic acid, cat gut, demineralized bone, and hydroxyapatite.

37. The method of claim 36, wherein said one or more substances are selected from the group consisting of gelatin, polyglycolic acid, and cat gut.

38. The method of claim 34, wherein said one or more substances is collagen.

39. The method of claim 38, wherein said collagen is bovine collagen.

40. The method of claim 38, wherein said collagen is porcine collagen type I or porcine collagen type III.

41. The method of claim 26, wherein said fibroblasts and said muscle cells are combined prior to said incubation.

42. The method of claim 26, wherein said fibroblasts and said muscle cells are added separately to said incubation.

43. The method of claim 26, wherein said incubating comprises: (1) culturing in culture medium comprising between 0.1% and about 20% human or non-human serum, followed by (2) culturing in serum-free culture medium.

44. The method of claim 26, wherein said incubating comprises culturing in serum-free medium.

45. The method of claim 26, wherein sufficient fibroblasts and muscle cells integrate within said biodegradable acellular matrix to substantially fill the space on and within said biodegradable acellular matrix available for cells.

46. A method for repairing tissue in a subject, wherein said method comprises:
(a) providing the composition of claim 7;
(b) identifying a site of tissue defect or tissue degeneration in said subject; and
(c) placing said composition at said site so that said tissue defect or degeneration is repaired.

47. The method of claim 46, wherein said tissue defect or tissue degeneration results in urinary incontinence, vesicoureteral reflux, or gastroesophageal reflux.

48. The method of claim 46, wherein said autologous fibroblasts are from gums, palate, skin, lamina propria, connective tissue, bone marrow, or adipose tissue of said subject.

49. The method of claim 46, wherein said autologous muscle cells are from the tongue, palatoglossus, temporalis muscle, soleus, gastrocnemius, or stemocleidomastoid muscle of said subject

50. A method for repairing a tissue defect in a subject, wherein said method comprises:
(a) providing a pharmaceutical composition comprising: (1) autologous, passaged fibroblasts, (2) autologous, passaged muscle cells, and (3) a pharmaceutically acceptable carrier thereof; wherein said pharmaceutical composition is substantially free of culture medium serum-derived proteins;
(b) identifying in said subject a site of tissue defect or tissue degeneration associated with a disorder selected from the group consisting of urinary incontinence, vesicoureteral reflux, and gastroesophageal reflux;
(c) injecting a therapeutically effective amount of said pharmaceutical composition adjacent to said site of tissue defect or degeneration, wherein said injecting results in repair of said tissue defect or degeneration.

51. The method of claim 50, wherein the step of providing a pharmaceutical composition comprises:
(a) providing a biopsy of fibroblast-containing tissue from said subject;
(b) separating fibroblasts from said biopsy so as to provide fibroblasts substantially free of extracellular matrix and non-fibroblast cells;
(c) culturing said fibroblasts under conditions that produce fibroblasts that are substantially free of culture medium serum-derived proteins;
(d) exposing said passaged fibroblasts to conditions that result in suspension of said fibroblasts;
(e) providing a muscle tissue biopsy from said subject;
(f) isolating muscle cells from said muscle tissue;
(g) culturing said muscle cells under conditions that produce muscle cells that are substantially free of culture medium serum-derived proteins;
(h) exposing said muscle cells to conditions that result in suspension of said muscle cells; and
(i) combining said fibroblast suspension with said muscle cell suspension and a pharmaceutically acceptable carrier to form said pharmaceutical composition.

52. The method of claim 51, wherein said fibroblast-containing tissue is selected from the group consisting of gums, palate, skin, lamina propria, connective tissue, bone marrow, and adipose tissue.

53. The method of claim 51, wherein said muscle tissue is selected from the group consisting of tongue, palatoglossus, temporalis muscle, soleus, gastrocnemius, and stemocleidomastoid muscle.

54. The method of claim 51, wherein said culturing of said fibroblasts or said muscle cells comprises: (1) culturing in a medium comprising between 0.1% and about 20% human or non-human serum, followed by (2) culturing in a serum-free medium.

55. The method of claim 51, wherein said culturing of said fibroblasts or said muscle cells comprises culturing in serum-free medium.

56. The method of claim 51, wherein said conditions that result in suspension of said fibroblasts or muscle cells comprise a proteolytic enzyme.

57. The method of claim 50, wherein said injecting comprises injecting a volume of said pharmaceutical composition into the urethra, or tissue adjacent to the urethra, of said subject such that the urethral lumen is compressed.

58. The method of claim 50, wherein said injecting comprises injecting a volume of said pharmaceutical composition into tissue adjacent to a ureteral orifice of said subject such that said orifice is compressed.

59. The method of claim 50, wherein said injecting comprises injecting a volume of said pharmaceutical composition into the tissue adjacent to the lower esophageal sphincter of said subject such that the esophagus is compressed.

60. An injectable composition for repairing tissue that has degenerated in a subject as a result of a disease, disorder, or defect in said subject, said injectable composition comprising:
(a) autologous, passaged fibroblasts and autologous, passaged muscle cells, wherein said fibroblasts and said muscle cells are substantially free of culture medium serum-derived proteins; and
(b) a biodegradable acellular injectable filler.

61. The injectable composition of claim 60, wherein said autologous fibroblasts are from gums, palate, skin, lamina propria, connective tissue, bone marrow, or adipose tissue of said subject.

62. The injectable composition of claim 60, wherein said autologous muscle cells are from the tongue, palatoglossus, temporalis muscle, soleus, gastrocnemius, or stemocleidomastoid muscle of said subject.

63. The injectable composition of claim 60, wherein said biodegradable acellular injectable filler, prior to combination with said fibroblasts and muscle cells, comprises one or more substances selected from the group consisting of (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from said subject.

64. The injectable composition of claim 63, wherein said one or more substances comprise an injectable dispersion of autologous collagen fibers.

65. The injectable composition of claim 64, wherein the concentration of said autologous collagen fibers in said injectable dispersion is at least 24 mg/ml.

66. The injectable composition of claim 63, wherein said one or more substances comprise collagen.

67. The injectable composition of claim 66, wherein said collagen is bovine collagen.

68. The injectable composition of claim 66, wherein said collagen comprises reconstituted bovine collagen fibers cross-linked with glutaraldehyde.

69. The injectable composition of claim 63, wherein said one or more substances are selected from the group consisting of solubilized gelatin, solubilized polyglycolic acid, and solubilized cat gut.

70. The injectable composition of claim 63, wherein said one or more substances comprise porcine gelatin powder and amino caproic acid dispersed in sodium chloride solution and an aliquot of plasma from said subject.

71. The injectable composition of claim 70, wherein the ratio of said sodium chloride solution and said aliquot of serum is 1:1 by volume.

72. The injectable composition of claim 71, wherein said sodium chloride solution comprises 0.9% sodium chloride by volume.

73. A method for making an injectable composition for repairing tissue that has degenerated in a subject as a result of a disease, disorder, or defect in said subject, wherein said method comprises:
(a) providing autologous, passaged fibroblasts and autologous, passaged muscle cells, wherein said fibroblasts and said muscle cells are substantially free of culture media serum-derived proteins;
(b) providing a biodegradable acellular filler; and
(c) combining said autologous, passaged fibroblasts, said autologous, passaged muscle cells, and said biodegradable acellular filler.

74. The method of claim 73, wherein said disease, disorder, or defect is associated with urinary incontinence, vesicoureteral reflux, or gastroesophageal reflux.

75. The method of claim 73, wherein said disease, disorder, or defect comprises defects of an oral mucosa, trauma to an oral mucosa, periodontal disease, diabetes, cutaneous ulcers, venous stasis, scars of skin, or wrinkles of skin.

76. The method of claim 73, wherein the step of providing autologous, passaged fibroblasts and autologous, passaged muscle cells comprises:
(a) providing a biopsy of fibroblast-containing tissue from said subject;
(b) separating autologous fibroblasts from said biopsy;
(c) culturing said autologous fibroblasts under conditions that result in fibroblasts that are substantially free of culture medium serum-derived proteins;
(d) exposing said incubated autologous fibroblasts to conditions that result in suspension of said fibroblasts;
(e) providing a biopsy of muscle tissue from said subject;
(f) isolating muscle cells from said muscle tissue biopsy;
(g) culturing said muscle cells under conditions that result in muscle cells that are substantially free of culture medium serum-derived proteins; and
(h) exposing said muscle cells to conditions that result in suspension of said muscle cells.

77. The method of claim 76, wherein said fibroblast-containing tissue is selected from the group consisting of gums, palate, skin, lamina propria, connective tissue, bone marrow, and adipose tissue.

78. The method of claim 76, wherein said muscle tissue comprises providing a biopsy from the tongue, palatoglossus, temporalis muscle, soleus, gastrocnemius, and stemocleidomastoid muscle.

79. The method of claim 76, wherein said culturing of said fibroblasts or said muscle cells comprises: (1) culturing in a medium comprising between 0.1% and about 20% human or non-human serum, followed by (2) culturing in a serum-free medium.

80. The method of claim 76, wherein said culturing of said fibroblasts or said muscle cells comprises culturing in serum-free medium.

81. The method of claim 76, wherein said culturing of said fibroblasts or said muscle cells is in a medium comprising a reagent that prevents the growth of mycoplasma.

82. The method of claim 81, wherein said reagent comprises tylosin.

83. The method of claim 82, wherein said reagent further comprises one or more compounds selected from the group consisting of gentamicin, ciprofloxacine, alatrofloxacine, azithromycin, and tetracycline.

84. The method of claim 76, wherein said conditions that result in suspension of said fibroblasts or muscle cells comprise a proteolytic enzyme.

85. The method of claim 73, wherein said biodegradable acellular filler, prior to combination with said fibroblasts and said muscle cells, comprises one or more substances selected from the group consisting of (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from said subject.

86. The method of claim 85, wherein said one or more substances comprise an injectable dispersion of autologous collagen fibers.

87. The method of claim 86, wherein the concentration of said autologous collagen fibers in said injectable dispersion is at least 24 mg/ml.

88. The method of claim 85, wherein said one or more substances comprise collagen.

89. The method of claim 88, wherein said collagen is bovine collagen.

90. The method of claim 88, wherein said collagen comprises reconstituted bovine collagen fibers cross-linked with glutaraldehyde.

91. The method of claim 85, wherein said one or more substances are selected from the group consisting of solubilized gelatin, solubilized polyglycolic acid, and solubilized cat gut.

92. The method of claim 85, wherein said one or more substances comprise porcine gelatin powder and amino caproic acid dispersed in sodium chloride solution and an aliquot of plasma from said subject.

93. The method of claim 92, wherein the ratio of said sodium chloride solution and said aliquot of serum is 1:1 by volume.

94. The method of claim 93, wherein said sodium chloride solution comprises 0.9% sodium chloride by volume.

95. A method for repairing tissue that has degenerated in a subject as a result of a disease, disorder, or defect in said subject, said method comprising injecting an effective amount of the composition of claim 60 into said subject at the site of said degeneration so that said tissue is repaired.

96. The method of claim 95, wherein said injecting comprises injecting a volume of said composition into the urethra or tissue adjacent to the urethra of said subject such that the urethral lumen is compressed.

97. The method of claim 95, wherein said injecting comprises injecting a volume of said composition into the tissue adjacent to the ureteral orifice of said subject such that said orifice is compressed.

98. The method of claim 95, wherein said injecting comprises injecting a volume of said composition into the tissue adjacent to the lower esophageal sphincter of said subject such that the esophagus is compressed.

99. The method of claim 95, wherein said biodegradable acellular injectable filler, prior to combination with said fibroblasts and muscle cells, comprises one or more substances selected from the group consisting of: (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from said subject.

100. The method of claim 99, wherein said one or more substances comprise collagen.

101. The method of claim 100, wherein said collagen is bovine collagen.

102. A method for repairing tissue that has degenerated in a subject as a result of a disease, disorder, or defect in said subject, said method comprising the steps of:
(a) injecting autologous, passaged fibroblasts into said subject at a site of tissue degeneration, wherein said fibroblasts are substantially free of culture medium serum-derived proteins;
(b) injecting autologous, passaged muscle cells into said subject at a site of a tissue defect or desired tissue augmentation, wherein said muscle cells are substantially free of culture medium serum-derived proteins; and
(c) injecting a biodegradable, acellular filler into the site, wherein said filler is substantially free of culture medium serum-derived proteins.

103. The method of claim 102, wherein each of said injecting steps (a)-(c) comprise injecting into the urethra or tissue adjacent to the urethra of said subject, wherein said method results in compression of the urethral lumen.

104. The method of claim 102, wherein each of said injecting steps (a)-(c) comprise injecting into the tissue adjacent to a ureteral orifice of said subject, wherein said method results in compression of said orifice.

105. The method of claim 102, wherein each of said injecting steps (a)-(c) comprise injecting into the tissue adjacent to the lower esophageal sphincter of said subject, wherein said method results in compression of the esophagus.

106. The method of claim 102, wherein said disease, disorder, or defect comprises defects of an oral mucosa, trauma to an oral mucosa, periodontal disease, diabetes, cutaneous ulcers, venous stasis, scars of skin, or wrinkles of skin.

107. The method of claim 102, wherein said autologous fibroblasts are from gums, palate, skin, lamina propria, connective tissue, bone marrow, or adipose tissue of said subject.

108. The method of claim 102, wherein said autologous muscle cells are from the tongue, palatoglossus, temporalis muscle, soleus, gastrocnemius, or stemocleidomastoid muscle of said subject.

109. The method of claim 102, wherein said fibroblasts and said muscle cells are injected simultaneously.

110. The method of claim 102, wherein said fibroblasts, said muscle cells, and said biodegradable acellular filler are injected simultaneously.

111. The method of claim 102, wherein said fibroblasts and muscle cells are injected separately.

112. The method of claim 102, wherein said fibroblasts and said muscle cells are injected separately from said biodegradable acellular filler.

113. The method of claim 112, wherein the duration between injecting said fibroblasts and said muscle cells into said subject and injecting said biodegradable acellular filler into said subject is about two weeks.

114. The method of claim 102, wherein said biodegradable acellular filler, prior to combination with said fibroblasts and said muscle cells, comprises one or more substances selected from the group consisting of: (a) an injectable dispersion of autologous collagen fibers; (b) collagen; (c) solubilized gelatin; (d) solubilized polyglycolic acid; (e) solubilized cat gut; and (f) porcine gelatin powder and amino caproic acid dispersed in sodium chloride solution and an aliquot of plasma from said subject.

115. The method of claim 114, wherein said one or more substances comprise an injectable dispersion of autologous collagen fibers.

116. The method of claim 115, wherein the concentration of said autologous collagen fibers in said injectable dispersion is at least 24 mg/ml.

117. The method of claim 114, wherein said one or more substances comprise collagen.

118. The method of claim 117, wherein said collagen is bovine collagen.

119. The method of claim 117, wherein said collagen comprises reconstituted bovine collagen fibers cross-linked with glutaraldehyde.

120. The method of claim 114, wherein said one or more substances are selected from the group consisting of solubilized gelatin, polyglycolic acid, and cat gut.

121. The method of claim 114, wherein said one or more substances comprise porcine gelatin powder and aminocaproic acid dispersed in sodium chloride solution, and an aliquot of plasma from the subject.

122. The method of claim 121, wherein the ratio of sodium chloride solution to said aliquot of serum is 1:1 by volume.

123. The method of claim 122, wherein said sodium chloride solution comprises 0.9% sodium chloride by volume.

124. The method of claim 102, wherein the ratio of autologous, passaged fibroblasts and autologous, passaged muscle cells to biodegradable, biodegradable acellular filler is approximately 1:1 by volume.

125. A device for repairing tissue that has degenerated in a subject as a result of a disease, disorder, or defect in said subject, said device comprising:
(a) a hypodermic syringe having a syringe chamber, a piston disposed therein, and an orifice communicating with said chamber; and
(b) a suspension comprising autologous, passaged fibroblasts, autologous, passaged muscle cells, and a pharmaceutically acceptable carrier, wherein said suspension is substantially free of culture medium serum-derived proteins, and wherein said suspension is disposed within said chamber.
